(19) United States
(12) Patent Application Publication
(10) Pub. No.: US 2006/0086362 A1
(43) Pub. Date: Apr. 27, 2006

(54) Intestinal ablation to limit food absorption

(76) Inventor: Stephen Solomon, New York, NY (US)

Correspondence Address: FRISHAUF, HOLTZ, GOODMAN & CHICK, P.C.;25th Floor, 767 Third Avenue, New York, NY, 10017-2023, US

(21) Appl. No.: 11/257,166

(22) Filed: Oct. 24, 2005

Related U.S. Application Data
(60) Provisional application No. 60/621,370, filed on Oct. 22, 2004.

(51) Int. Cl. A61B 18/08

(52) U.S. Cl. 128/898; 606/31

(57) ABSTRACT

A method for treating obesity in a human patient is provided whereby an ablation source is introduced into the small intestine of the patient via a working channel of an endoscope, and the intestinal mucosa of the patient is ablated over a length of the small intestine in a range of approximately 40 cm to 100 cm.

### FIELD OF THE INVENTION

The present invention relates to a method and apparatus for treating obesity by ablating intestinal mucosa to cause malabsorption.

### BACKGROUND OF THE INVENTION

Intestinal villi are invaginations in the intestinal mucosa that maximize surface area to enable food absorption. Diseases such as celiac sprue cause the villi to atrophy. As a result of the atrophy of the villi, the ability of the intestine to absorb food is decreased, which is known as malabsorption. Malabsorption is known to lead to weight loss.

### SUMMARY OF THE INVENTION

According to one aspect of the invention, a method for causing weight loss is provided that includes ablating intestinal mucosa over a length of small intestine in a patient.

### BRIEF DESCRIPTION OF THE DRAWINGS

FIG. 1 shows the introduction of an ablation apparatus via an endoscope;

FIG. 2 is a detailed view of the ablation apparatus in use to ablate intestinal mucosa; and

FIG. 3 shows an example of a heating element used in the ablation apparatus.

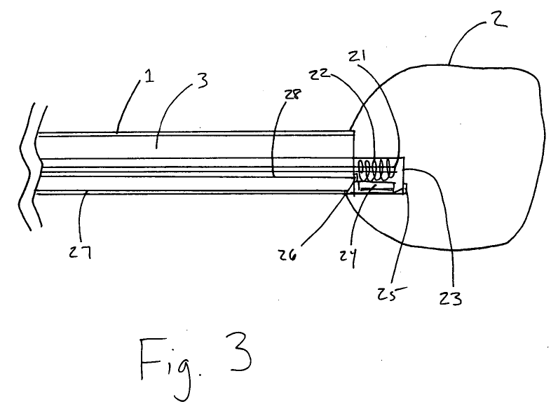

### DETAILED DESCRIPTION

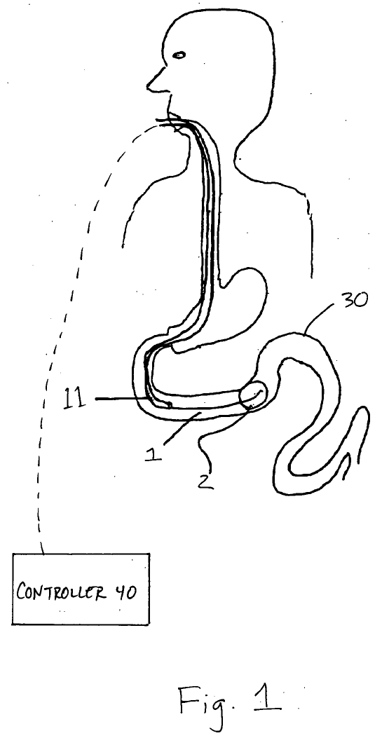

As shown in FIG. 1, an endoscope will be orally inserted and guided to the small intestine. An apparatus for ablating the intestinal mucosa will be introduced through a working channel of the endoscope, and the intestinal mucosa will be ablated over a desired length of the small intestine. The ablation should be performed to be focused on the absorptive villi of the mucosa, and should not be transmural, so as to avoid weakening the intestinal wall.

The length of intestine that is ablated will vary in accordance with the amount of malabsorption to be effected. The ablated length of the intestine could, for example, be in the range of approximately 40-100 cm. Or the ablated length of the intestine could be significantly more or significantly less.

Typically, in an otherwise healthy individual the villi and mucosa will regenerate so that the obesity therapy is temporary.

The ablation may be performed thermally or chemically, and the ablation apparatus may be one of several known devices used to ablate esophageal mucosa, for example in treating Barrett's esophagus. Specifically, a thermal ablation device (for multipolar electrocoagulation), a photothermal ablation device or a cryotherapy device could be used to ablate the intestinal mucosa. A mechanical ablation device could also be used.

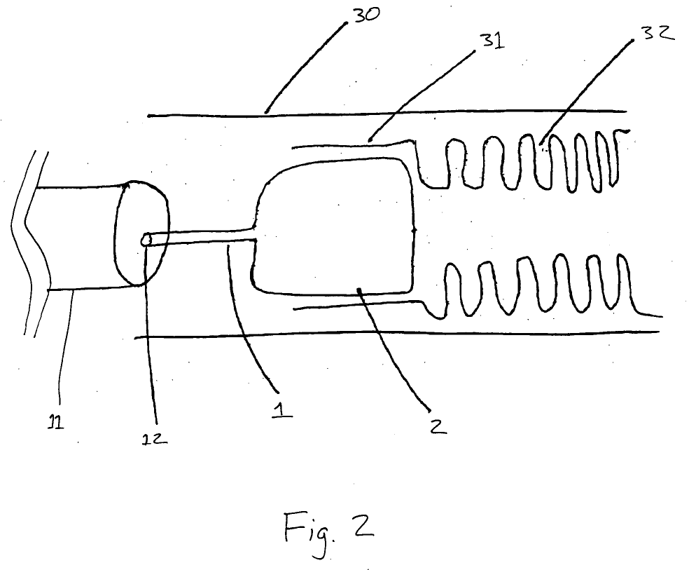

One example of the ablation apparatus is shown in FIG. 2. Specifically, the apparatus includes an ablation catheter 1 inserted through the working channel 12 of an endoscope 11. An inflatable balloon 2 (distensible bladder) is provided at the distal end of the catheter 1. The balloon 2 is inflatable with a liquid through a lumen 3 of the catheter 1.

A heating element will be secured to the distal end of the catheter and situated within the balloon 2. The heating element will include a tubular core member 21 secured to the distal end of the catheter. A resistive wire 22 will be wound around the core member 21 and a tubular heat conductive shell 23 will be disposed over the core member 21 and resistive wire 22. The shell may be formed with a wall having a plurality of slots 24 for permitting the liquid inflating the balloon 2 to contact the resistive wire 22. A first thermocouple 25 may be secured to an outside surface of the wall of the tubular shell 23 and a second thermocouple 26 may be secured to an inside surface of the wall of the tubular shell 23.

The distal end of the catheter 1 will be guided to the farthest point in the small intestine 30 that is to be ablated. The balloon 2 may be inflated to press against the wall of the small intestine 30 and the heating element may be controlled to heat the liquid in the balloon 2. The liquid in the balloon 2 should be heated so as to heat the intestinal mucosa to between 60-100° C., while maintaining a temperature of less than 37° C. at the external wall of the intestine. The inflated balloon 2, with the heated liquid therein, will then dragged backwards toward the end of the endoscope 11, so as to ablate the mucosa that contacts the inflated, heated balloon 2. See the ablated epithelium 31 in FIG. 2.

The villi 32 that do not contact the inflated, heated balloon 2 will remain unaffected.

The balloon 2 may be deflated to be withdrawn into the working channel of the endoscope 11 while the endoscope and ablation apparatus are removed from the patient.

A controller 40 may be provided to control and supply power to the heating element. The thermocouples 25 and 26 can provide feedback to the controller 40 via leads 27 and 28 to allow temperature control of the fluid within the balloon 2.

In addition, the liquid could be heated externally and delivered to the balloon 2 while heated, thereby avoiding the need for a heating element within the balloon 2. With this structure, the catheter 1 should be insulated.

Alternatively, the intestinal mucosa could be ablated using radiofrequency or microwave energy generated at a core housed within the balloon. With this structure, the fluid used to inflate the balloon should conduct the energy generated at the core.

Still further, cold liquid in the balloon 2 could be used instead of hot liquid to perform the ablation. Alternatively, a cryogenic spray could be used to ablate the intestinal mucosa. Alternatively, cryogens contained within a closed system could be allowed to expand according to the Joule-Thompson principle to create ice that would freeze the mucosa.

Although the ablation method described above is performed via an endoscope, the ablation could be performed using the ablation apparatus with fluoroscopic guidance without the endoscope. In addition, both fluoroscopic and endoscopic guidance could be used. For example, the endoscope could be used to introduce the ablation apparatus to the duodenum, while, if desired, the ablation apparatus could be advanced farther into the small intestine under fluoroscopic guidance.

The foregoing method may be utilized to treat obese human patients and/or merely overweight human patients who desire to achieve weight loss. And, of course, the method may also be applied to other non-human mammals.

Various modifications and additions can be made without departing from the spirit and scope of the invention. Accordingly, the foregoing description is meant to be taken only by way of example and not to otherwise limit the scope of the present invention as defined in the appended claims.

What is claimed is:

1. A method for causing weight loss in a patient in need thereof, comprising:
ablating intestinal mucosa of the patient over a length of small intestine.

2. The method according to claim 1, wherein the ablation is performed using one of a thermal ablation source and a chemical ablation source.

3. The method according to claim 2, wherein the ablation source is introduced into the small intestine via a working channel of an endoscope.

4. The method according to claim 3, wherein the ablation source is fluoroscopically visualized while performing the ablation.

5. The method according to claim 1, wherein the length of small intestine ablated is in a range of approximately 40 cm to 100 cm.

6. A method for treating obesity in a human patient, comprising:
introducing an ablation source into the small intestine of the patient via a working channel of an endoscope;
ablating intestinal mucosa of the patient over a length of the small intestine in a range of approximately 40 cm to 100 cm.
